(19) United States
(12) Patent Application Publication
(10) Pub. No.: US 2025/0281075 A1
(43) Pub. Date: Sep. 11, 2025

(54) TRANSDERMAL GLUCOSE MONITORING SYSTEM

(71) Applicant: BIONIME CORPORATION, Taichung (TW)

(72) Inventors: Chun-Mu HUANG, Taichung (TW); Chihlung YEH, Taichung (TW); Chengwei LU, Taichung (TW); Hungfa SUN, Taichung (TW); Tingyu LIU, Taichung (TW); Yuhan CHANG, Taichung (TW); Yenhsin HUANG, Taichung (TW)

(73) Assignee: BIONIME CORPORATION, Taichung (TW)

(21) Appl. No.: 19/070,565

(22) Filed: Mar. 5, 2025

Related U.S. Application Data
(60) Provisional application No. 63/561,355, filed on Mar. 5, 2024.

(51) Int. Cl. A61B 5/145; A61B 5/00; A61B 5/1495; G16H 10/60; G16H 15/00

(52) U.S. Cl. CPC A61B 5/14532; A61B 5/14503; A61B 5/1451; A61B 5/1495; A61B 5/6867; A61B 5/7264; A61B 5/7275; A61B 5/7282; A61B 5/743; A61B 5/746; A61B 5/7475; G16H 10/60; G16H 15/00; A61B 2560/0214; A61B 2560/0223; A61B 2560/0242; A61B 2560/045; A61B 2560/0462; A61B 2562/0271

(57) ABSTRACT

A transdermal glucose monitoring system is provided. The system includes: a continuous glucose monitoring (CGM) device, configured to monitor glucose concentration in subcutaneous interstitial fluid of a patient; a patient user interface, configured to acquire information of the CGM device, receive glucose concentration data, and display a warm-up progress indicator of the CGM device; and a cloud server, configured to complete data sharing and authorize access to data. The provided patient user interface focuses on the warm-up time of the CGM device and provides a positive and intuitive display thereof, along with visual management of glucose monitoring data analysis, thereby enhancing the convenience of glucose monitoring. Furthermore, the system supports a second monitoring device, enabling family and friends to promptly understand the patient's glucose monitoring status and infer the causes of glucose fluctuations.

### CROSS REFERENCE TO THE RELATED APPLICATIONS

This application is based upon and claims priority to American Patent Application No. 63/561,355, filed on Mar. 5, 2024, the entire contents of which are incorporated herein by reference.

### TECHNICAL FIELD

The present disclosure relates to the technical field of medical detection, and more particularly, to a transdermal glucose monitoring system.

### BACKGROUND

Diabetes mellitus is a systemic metabolic disorder often accompanied by abnormalities in lipid and protein metabolism. Chronic hyperglycemia may lead to common complications including diabetic retinopathy, diabetic nephropathy, and diabetic foot syndrome. For individuals with diabetes, blood glucose monitoring holds significant clinical relevance. Proper execution of blood glucose surveillance not only enhances the efficacy of diabetes management and disease progression tracking, but also serves critical functions in evaluating therapeutic intervention outcomes and facilitating evidence-based modifications to nutritional protocols, physical activity regimens, and pharmacological treatment plans.

Daily blood glucose monitoring generally uses venous blood glucose monitoring and fingertip blood glucose monitoring, wherein, venous blood glucose monitoring may detect fasting blood glucose, two-hour postprandial blood glucose, and understand the blood glucose control in the last three months; and fingertip blood glucose monitoring may help diabetic patients monitor blood glucose at any time. With the advent of continuous glucose monitoring, patients may now monitor the glucose concentration in the interstitial fluid beneath the skin via a glucose sensor, which indirectly reflects blood glucose levels, allowing for the plotting of glucose fluctuation curves, providing continuous, comprehensive, and reliable 24-hour glucose information, and enabling the detection of hyperglycemic and hypoglycemic events that are often undetectable by traditional monitoring methods.

Currently, commercially available continuous glucose monitoring (CGM) systems for monitoring a patient's blood glucose/glucose concentration (levels) comprise a sensor and a transmitter. A sensor, after being implanted subcutaneously, detects physiological signals in the interstitial fluid and transmits these signals to a transmitter, which is capable of issuing an alert when abnormal physiological signals are detected. However, before the sensor can accurately measure the physiological signals, it must be fully “wetted” or hydrated to achieve equilibrium with the glucose in the patient's body. Therefore, upon subcutaneous implantation of the sensor, a warm-up period is necessary before the sensor initiates measurement, enabling the patient/user to acquire accurate glucose concentration readings. In existing continuous glucose monitoring systems, the graphical user interface (GUI) and navigation process at the patient/user end lack sufficient consideration for an intuitive representation of the device warm-up period. Moreover, current systems are insufficient in improving blood glucose control efficacy and lack the capability to promptly determine the reasons for blood glucose variations among family members. To address this issue, a more intuitive navigation design would facilitate users in interpreting analyte measurement results with greater accuracy, which represents a significant technical challenge urgently requiring resolution in the current field of technology.

### SUMMARY

In view of this, the present disclosure provides a transdermal glucose monitoring system, to address the problems identified in the background art.

In order to achieve the above effects, the present disclosure adopts the following technical solutions.

A transdermal glucose monitoring system comprises:
a continuous glucose monitoring (CGM) device, configured to monitor glucose concentration in subcutaneous interstitial fluid of a patient;
a patient user interface, configured to acquire information of the CGM device, receive glucose concentration data, and display a warm-up progress indicator of the CGM device; and
a cloud server, configured to complete data sharing and authorize access to data;
wherein, a user interface of the patient user interface comprises:
a top information section, configured to provide remaining usage days, a real-time glucose value and trend indicators of the current CGM device, and visually indicate glycemic status through color variations or graphical symbols;
a glucose trend line section, configured to graphically display glucose trend data over a past predetermined number of hours;
a daily monitoring record overview chart section, configured to display daily glucose trend patterns in the patient over a multi-day period;
a time in range (TIR) distribution chart section, configured to provide statistical data for glucose management, display a percentage of time for the patient's glucose values within a target glucose range, and visually represent a temporal distribution of glucose levels via color-coded bar charts; and
a bottom navigation bar, configured to provide functional buttons, to enable the patient to quickly access different functions of an application program.

In the transdermal glucose monitoring system, optionally, the CGM device comprises:
a glucose sensor, being implanted subcutaneously into the patient, configured to monitor glucose concentration in subcutaneous interstitial fluid of the patient;
a temperature sensor, configured to monitor environmental temperature;
a glucose measurement unit, configured to apply a voltage to the glucose sensor to measure glucose concentration;
a battery, configured to supply power; and
a wireless communication unit, configured to transfer data.

In the transdermal glucose monitoring system, optionally, the patient user interface comprises:
a user interface, configured to provide a warm-up status display, a glucose data display and a report analysis function;
a wireless communication module, configured to receive glucose concentration data transmitted by the CGM device;
a memory, configured to store the glucose concentration data, trend analysis, health records of the patient, and calibration data;
one or more processors, configured to perform multiple functional instructions;
a warm-up control unit, configured to provide the warm-up progress indicator and completion time of the CGM device via one or more processors; and
a glucose data processing unit, configured to, upon completion of the warm-up, continuously receive and process the glucose concentration data via one or more processors, generate real-time glucose reading values, dynamic glucose fluctuation curves and glucose management reports.

In the transdermal glucose monitoring system, optionally, the warm-up control unit comprises:
a time setting module, configured to enable one or more processors to provide a scheduled warm-up completion time for the CGM device based on a warm-up preset time;
a response module, configured to enable one or more processors to monitor a power-on action of the CGM device and to respond thereto; and
a time recording module, configured to cause one or more processors to record a power-on time and an elapsed warm-up time of the CGM device.

In the transdermal glucose monitoring system, optionally, the warm-up control unit comprises:
a power check module, configured to monitor remaining battery levels of the CGM device and the patient user interface, compare the remaining battery levels with a preset activation threshold, and determine whether to initiate power-on and warm-up procedures; and
a time setting module, configured to cause one or more processors to generate a scheduled completion time of warm-up for the CGM device based on a warm-up preset time.

In the transdermal glucose monitoring system, optionally, the patient user interface comprises:
an annotation module, configured to record medication status, diet status, and exercise status of the patient, and obtain user information of the patient according to a log record;
a warm-up progress display module, configured to display the warm-up progress of the CGM device; and
a glucose display module, configured to display current glucose concentration data and glucose value fluctuations according to different types of trend indicators in a block design.

In the transdermal glucose monitoring system, optionally, a display mode of the warm-up progress display module comprises: the elapsed warm-up time and/or the scheduled completion time of warm-up.

In the transdermal glucose monitoring system, the patient user interface further comprises a calibration unit, optionally, the calibration unit comprises:
a blood glucose data acquisition module, configured to receive blood glucose measurement values obtained from a patient via a blood glucose monitor (BGM), and be autonomously input by the patient or automatically transmitted to the patient user interface;
a determining module, configured to analyze the validity of the blood glucose data and confirm whether the CGM device operates normally; and
a calibration unit, configured to write the blood glucose data into the patient user interface when the blood glucose data is valid and the CGM device operates normally.

In the transdermal glucose monitoring system, optionally, the patient user interface further comprises:
a drawing module, configured to perform glucose curves drawing and TIR metrics statistics according to the glucose concentration data of the patient;
an adjusting module, configured to correct the glucose curves and the TIR metrics based on the glucose concentration data; and
a generation module, configured to generate a CGM standard report and an ambulatory glucose profile report for the glucose concentration data.

In the transdermal glucose monitoring system, optionally, the glucose curves include a single-day glucose curve graph, a multi-day continuous glucose monitoring thumbnail graph, and a multi-day quartile graph.

In the transdermal glucose monitoring system, optionally, the glucose curves and the TIR metrics diagram show the glucose status of the patient through different colors.

In the transdermal glucose monitoring system, optionally, the patient user interface further comprises an alarm module, configured to send alarm information to the patient user interface when a glucose value of the patient deviates from a healthy glucose value range.

A transdermal glucose monitoring system comprises:
a partnership establishment module, configured to authorize a connection by scanning, via a camera, barcode information associated with the CGM device, or configured to transmit an invitation link to the patient user interface via a third-party platform, thereby completing authentication of a patient identity and a partner identity as well as authorization; and
a partner user interface, configured to query glucose concentration data, as well as the patient's basic information, glucose monitoring orders, medication orders, and consultation orders, based on the patient's authorization.

In the transdermal glucose monitoring system, optionally, the partner user interface comprises:
an alert module, configured to monitor glucose fluctuations of multiple partners to enable timely and effective interventions;
an update module, configured to prioritize and observe real-time updates of a specific partner's glucose monitoring status;
a glucose display module, configured to acquire current glucose monitoring statuses of multiple partners and infer potential causes of fluctuations based on trends in glucose changes; and
a blood glucose display module, configured to acquire the most recent blood glucose measurement records.

In the transdermal glucose monitoring system, optionally, the partner user interface further comprises an annotation classification module, configured to record glucose anomaly events and their occurrence times, mark a range of glucose anomalies, and convert a marked content onto a timeline for comparing glucose fluctuations, thereby inferring potential causes of the glucose fluctuations.

In the transdermal glucose monitoring system, optionally, the partner user interface further comprises a glycated hemoglobin display module, which is configured to map a patient's glycated hemoglobin value onto a color-coded chart, thereby enabling direct identification of the specific range within a commonly recognized standard system where the patient's glycated hemoglobin result falls.

Based on the aforementioned technical solutions, it can be concluded that, compared to the prior art, the present disclosure discloses a transcutaneous glucose monitoring system, which focuses on the warm-up time of the CGM device and provides a positive and intuitive display, and grants patients the right to perform background calibration using blood glucose data, assisting the patients' family members in promptly understanding the glucose monitoring status of their relatives, inferring the causes of glucose fluctuations, and facilitating subsequent diagnosis and treatment, thereby further enhancing the effectiveness of glucose control.

### BRIEF DESCRIPTION OF THE DRAWINGS

In order to more clearly illustrate the embodiments of the present disclosure or the technical solutions in the prior art, the drawings required in the description of the embodiments or the prior art will be briefly described below, and obviously, the drawings in the following description are merely embodiments of the present disclosure, and for those of ordinary skill in the art, other drawings may be obtained according to the provided drawings without creative efforts.

FIG. 1 is a schematic composition diagram of a transdermal glucose monitoring system according to Embodiment 1;

FIG. 2 is a schematic composition diagram of a continuous glucose monitoring (CGM) device according to Embodiment 1;

FIG. 3 is a schematic composition diagram of a patient user interface according to Embodiment 1;

FIG. 4 is a schematic composition diagram of a warm-up control unit according to Embodiment 1;

FIG. 5 is a schematic diagram of use of an annotation module according to Embodiment 1;

FIG. 6 is an interface display diagram of a patient user interface according to Embodiment 1;

FIG. 7 is a display mode of a warm-up progress display module according to Embodiment 1;

FIG. 8 is a structural diagram of a calibration unit according to Embodiment 1;

FIG. 9 is a structural diagram of a cloud server according to Embodiment 1;

FIG. 10 is a structural diagram of a transdermal glucose monitoring system according to Embodiment 2;

FIG. 11 is an interface display diagram of a partner user interface according to Embodiment 2;

FIG. 12 is another interface display diagram of a partner user interface according to Embodiment 2;

FIG. 13 is an interface diagram of a glycated hemoglobin display module according to Embodiment 2.

### DETAILED DESCRIPTION OF THE EMBODIMENTS

In the following, the technical solutions in the embodiments of the present disclosure will be clearly and completely described with reference to the drawings in the embodiments of the present disclosure. Obviously, the described embodiments are only a part of the embodiments of the present disclosure, but not all the embodiments thereof. Based on the embodiments of the present disclosure, all other embodiments obtained by those skilled in the art without any creative efforts shall fall within the scope of the present disclosure.

### Embodiment 1

An embodiment of the present disclosure discloses a transcutaneous glucose monitoring system, as shown in FIG. 1, comprising:
a continuous glucose monitoring (CGM) device 2, configured to monitor glucose concentration in subcutaneous interstitial fluid of a patient;
a patient user interface 3, configured to acquire information of the CGM device, receive glucose concentration data, and display a warm-up progress indicator of the CGM device 2; and
a cloud server 4, configured to complete data sharing and authorize access to data.

Based on the structure shown in FIG. 1, the transcutaneous glucose monitoring system (1) of the present disclosure comprises a CGM device (2), including a glucose sensor 21 configured to be implanted subcutaneously in a patient, and a wireless communication unit (transmitter) 22 configured to transmit glucose signals or data sensed by the glucose sensor 21; and a patient user interface 3, which is a mobile phone or receiver equipped with a relevant application (APP) installed, configured to provide real-time glucose values, dynamic glucose fluctuation curves, and glucose management reports, thereby enabling users to intuitively understand the overall glucose profile and the causes of glucose variability, and further improve lifestyle and health care. In addition, the patient user interface 3 is wirelessly connected to a cloud server 4 to upload data. The cloud server 4 is configured to provide an authorization mechanism, allowing a partner user interface 5 or a medical care interface 6 to access the patient's glucose data in real time, thereby enabling remote collaborative care and facilitating real-time monitoring and emergency response. The cloud server 4 is configured to utilize an encryption mechanism to ensure the security of data transmission and provide historical data analysis functionality, thereby assisting patients and medical personnel in long-term glucose management.

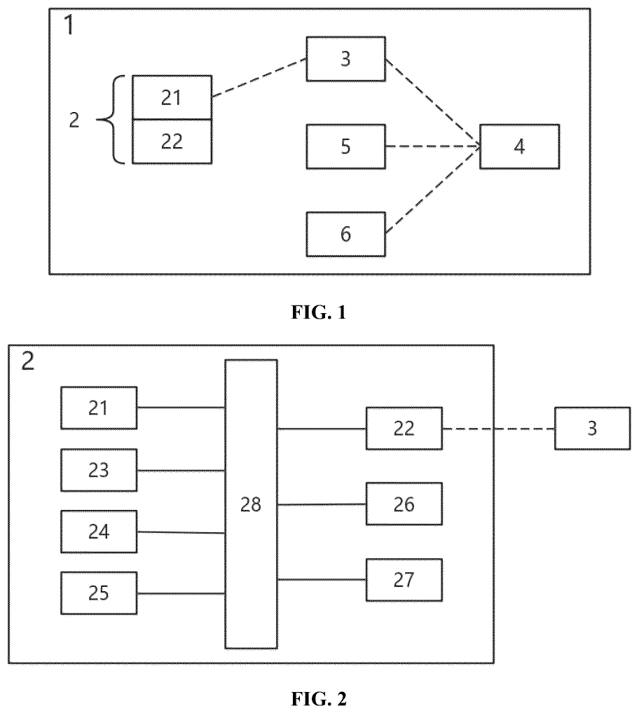

Further, as shown in FIG. 2, the CGM device 2 of the present embodiment includes:
a glucose sensor 21, being implanted subcutaneously into the patient, configured to monitor glucose concentration in subcutaneous interstitial fluid of a patient;
a temperature sensor 23, configured to monitor environmental temperature;
a glucose measurement unit 24, configured to apply a voltage to the glucose sensor to measure glucose concentration;
a battery 26, configured to supply power; and
a wireless communication unit 22, configured to transfer data; and
a memory 27, configured for data storage.

In another embodiment, the CGM device 2 further includes an acceleration sensor 25, configured to detect motion or posture of the patient and provide motion information and sleep information. The acceleration sensor 25 is used to determine measurement errors caused by changes in glucose levels and changes in motion or posture, thereby improving the accuracy and reliability of the CGM device 2.

In the present embodiment, the CGM device 2, as shown in FIG. 2, is configured to continuously and dynamically monitor glucose changes. Within the lifecycle of the glucose sensor 21, the measurement frequency of glucose may be set to every 30 seconds, every minute, every 2 minutes, every 5 minutes, every 10 minutes, every 15 minutes, every 30 minutes, or every hour. Then, the wireless communication unit 22 is configured to transmit glucose signals measured by the glucose sensor 21 (or, depending on the design of the transmitter and receiver for signal conversion and sensing functions, convert the glucose signals into glucose data) to the patient user terminal 3, which is then displayed on the user interface. In another embodiment, the glucose sensor 21 may be combined with a miniature transmitter and implanted subcutaneously (not shown in the figures).

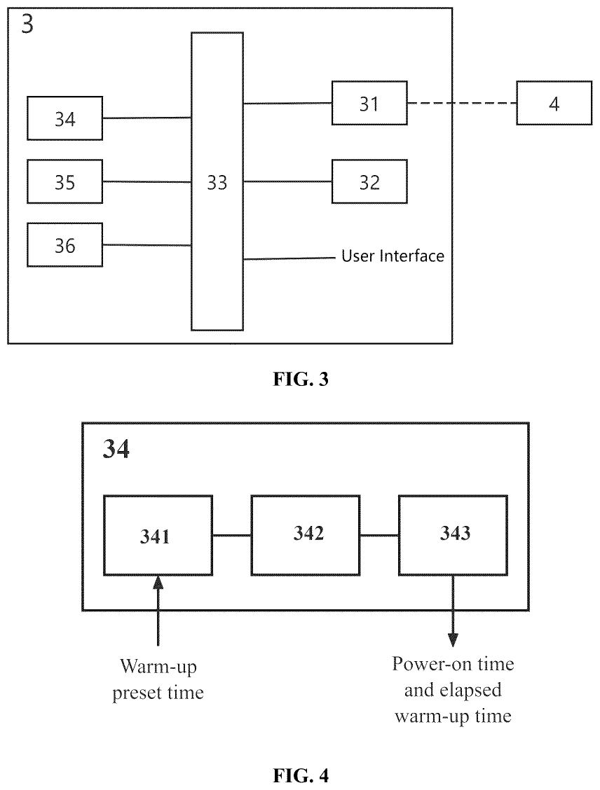

Further, as shown in FIG. 3, the patient user interface 3 comprises:
a user interface, configured to provide a warm-up status display, a glucose data display and a report analysis functions;
a wireless communication module 31, configured to receive glucose concentration data transmitted by the CGM device;
a memory 32, configured to store the glucose concentration data, trend analysis, health records of the patient, and calibration data;
one or more processors 33, configured to perform multiple functional instructions;
a warm-up control unit 34, configured to provide the warm-up progress indicator and completion time of the CGM device via one or more processors; and
a glucose data processing unit 35, configured to, upon completion of the warm-up, continuously receive and process the glucose concentration data via one or more processors, generate real-time glucose reading values, dynamic glucose fluctuation curves and glucose management reports.

In the present embodiment, the patient user interface 3 is wirelessly connected to the cloud server 4 to upload data. Through the authorization mechanism provided by the cloud server 4, the partner user interface 5 or the medical care interface 6 may obtain the patient's glucose data in real time for remote collaborative care, so as to enable real-time monitoring and emergency response. The cloud server 4 may utilize an encryption mechanism to ensure the security of data transmission and provide historical data analysis functionality to assist patients and medical personnel in long-term glucose management.

Further, as shown in FIG. 4, the warm-up control unit 34 of the present embodiment comprises:
a time setting module 341, configured to enable one or more processors 33 to provide a scheduled warm-up completion time for the CGM device 2 based on a warm-up preset time;
a response module 342, configured to enable one or more processors 33 to monitor a power-on action of the CGM device 2 and to respond thereto; and
a time recording module 343, configured to cause one or more processors 33 to record a power-on time and an elapsed warm-up time of the CGM device 2.

In another embodiment, the warm-up control unit 34 comprises:
a power check module, configured to monitor remaining battery levels of the CGM device 2 and the patient user interface 3, compare the remaining power with a preset activation threshold, to determine whether to initiate power-on and warm-up procedures; and when the remaining power of the CGM device 2 is below a startup threshold, the startup process is automatically delayed, and a low power warning is provided until the power is sufficient to execute a warm-up procedure;
a time setting module, configured to cause one or more processors 33 to generate a scheduled completion time of warm-up for the CGM device 2 based on a preset warm-up duration.

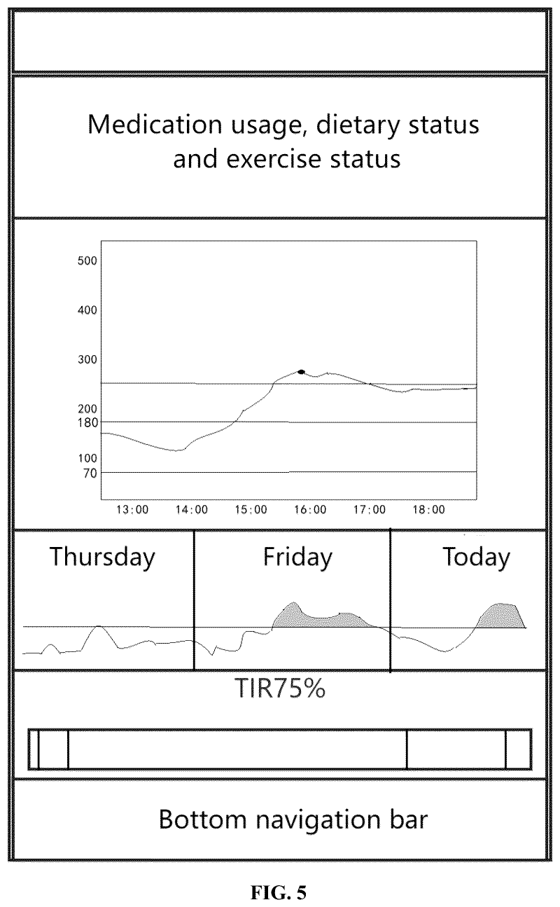

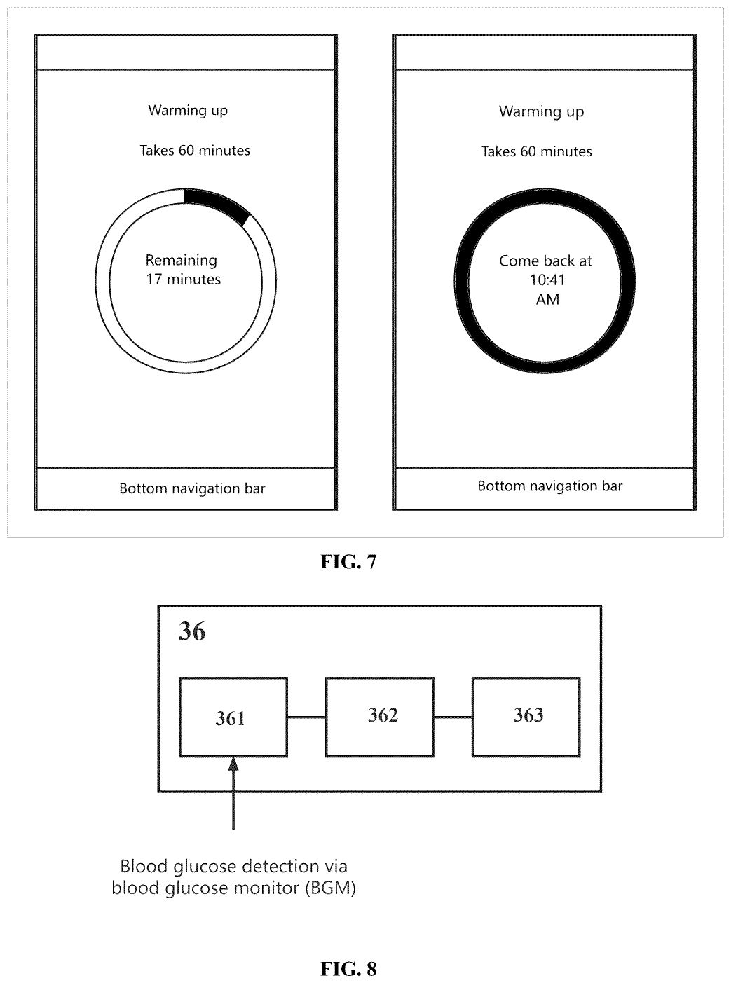

Further, the user interface of the patient user interface 3 in the present embodiment includes:
an annotation module, configured to record medication status, diet status, and exercise status of the patient, and obtain user information of the patient according to a log record, referring to FIG. 5;
a warm-up progress display module, configured to display the warm-up progress of the CGM device, referring to FIG. 7; and
a glucose display module, configured to display current glucose concentration data and glucose value fluctuations according to different types of trend indicators in a block design.

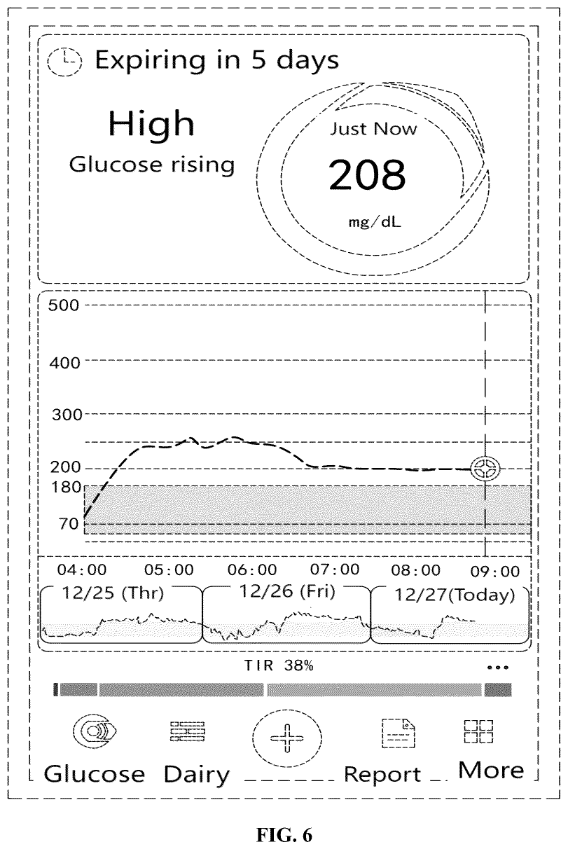

As shown in FIG. 6, the user interface of the patient interface 3 is illustrated. The block-based design of the interface creates a sense of spaciousness, enhancing the cleanliness and comfort of the interface. The interface is configured to visually display glucose information, assisting the user in real-time monitoring and analysis of glucose trend changes, thereby facilitating more effective diabetes management. The block design of the graphical user interface (GUI) allows users to quickly access essential information and further adjust diet, exercise, or medication usage to maintain stable glucose levels. The GUI primarily includes:
a top information section, configured to provide remaining usage days, a real-time glucose value and trend indicators of the current CGM device, and visually indicate glycemic status through color variations or graphical symbols; for example, the system is configured to alert the user by displaying different colors (e.g., green, orange, or red) in response to abnormal glucose level. Furthermore, the section is configured to display glucose trend information, indicating whether the current glucose level is in a rising, falling, or stable state, thereby enabling the user to take early action in response to changes in glucose levels. In some embodiments, the trend indicators are selectively replaceable with other types of icons, wherein variations in color and the directional orientation of the trend indicators are configured to rapidly highlight distinct abnormal conditions;
a glucose trend line section, configured to graphically display glucose trend data over a past predetermined number of hours (such as 5 hours); in the graphical representation, the horizontal axis is configured to represent time, and the vertical axis is configured to represent glucose values, wherein different colors are employed to demarcate glucose ranges, for instance, green for a normal range, and yellow and red for hyperglycemic and hypoglycemic states, respectively. The chart is configured to facilitate the visualization of glucose fluctuations and offers interactive functionality, allowing the user to click on a specific time point to access detailed glucose data, thus enabling further investigation into potential factors influencing glucose variations;
a daily monitoring record overview chart section, configured to display daily glucose trend patterns in the patient over a multi-day period (such as 3 days), enabling the patient to quickly understand glucose variations across multiple days. The chart automatically highlights color-coded blocks for glucose levels greater than 180 mg/dL and less than 70 mg/dL, allowing the patient to identify patterns in glucose fluctuations and adjust dietary, exercise, or medication plans to optimize glucose control, the daily monitoring record overview chart section presented in multiple daily glucose trend overview chart frames, each displaying hyperglycemic and hypoglycemic marker areas to facilitate user review of blood glucose fluctuations over the past several days, enabling appropriate corrective actions;
a time in range (TIR) distribution chart section, configured to provide statistical data for glucose management, wherein the TIR value displays a percentage of time for the patient's glucose values within a target glucose range, and visually represents a temporal distribution of glucose levels via color-coded bar charts; and for example, green indicates that glucose levels are within the normal range, while yellow or red respectively indicate that glucose levels are elevated or reduced. This section assists the user in evaluating the stability of their glucose control and making necessary adjustments to mitigate the risk of long-term glucose abnormalities; and
a bottom navigation bar, configured to provide functional buttons, to enable the patient to quickly access different functions of an application program. Wherein, the buttons include: glucose data viewing, log recording, report generation, and other application functions or setting options. The log recording function is configured to allow users to record diet, exercise, and insulin usage, thereby providing comprehensive glucose management information; and the report generation function is configured to organize glucose data into charts and reports, facilitating analysis by users or medical professionals. Additionally, the buttons in the bottom navigation bar are customizable based on user preferences, thereby enhancing operational efficiency.

In the present embodiment, the intuitive and user-friendly interface is configured to assist users in quickly accessing and analyzing glucose information, thereby improving the accuracy and personalization of glucose monitoring, which contributes to enhancing the efficiency and effectiveness of diabetes management.

As shown in FIG. 7, a display mode of the warm-up progress display module is: the elapsed warm-up time and/or the scheduled completion time of warm-up. Compared to the countdown mechanism for warm-up used in conventional products on the market, the present design adopts a positive indication or directly displays the scheduled completion time of the warm-up process, thereby enabling the user to more intuitively understand when the warm-up process will be completed.

Further, as shown in FIG. 8, the patient user interface 3 of the present embodiment further comprises a calibration unit 36, optionally, the calibration unit 36 comprises:
a blood glucose data acquisition module 361, configured to receive blood glucose measurement values obtained from a patient via a blood glucose monitor (BGM), and be autonomously input by the patient or automatically transmitted to the patient user interface 3;
a determining module 362, configured to analyze the validity of the blood glucose data and confirm whether the CGM device 2 operates normally; and
a calibration unit 363, configured to write the blood glucose data into the patient user interface 3 when the blood glucose data is valid and the CGM device 2 operates normally.

In the present embodiment, the calibration unit 36 (as illustrated in FIG. 8) is configured to calibrate real-time collected glucose data based on blood glucose data obtained from a blood glucose monitor, thereby mitigating deviations in continuous glucose monitoring. When the collected blood glucose data is valid and the CGM device 2 is operating normally, the blood glucose data is written as calibration data into the patient user interface 3 and stored therein. An authorized user is enabled to access historical data on-demand, wherein secure backup of the data prevents loss of glycemic information due to device malfunction, misplacement, or damage, such that data recovery or reconstruction remains achievable even under contingency scenarios.

In another embodiment, the patient user interface 3 incorporates a blood glucose monitoring module, configured to directly perform blood glucose detection and calibration operations without requiring additional external blood glucose monitoring devices.

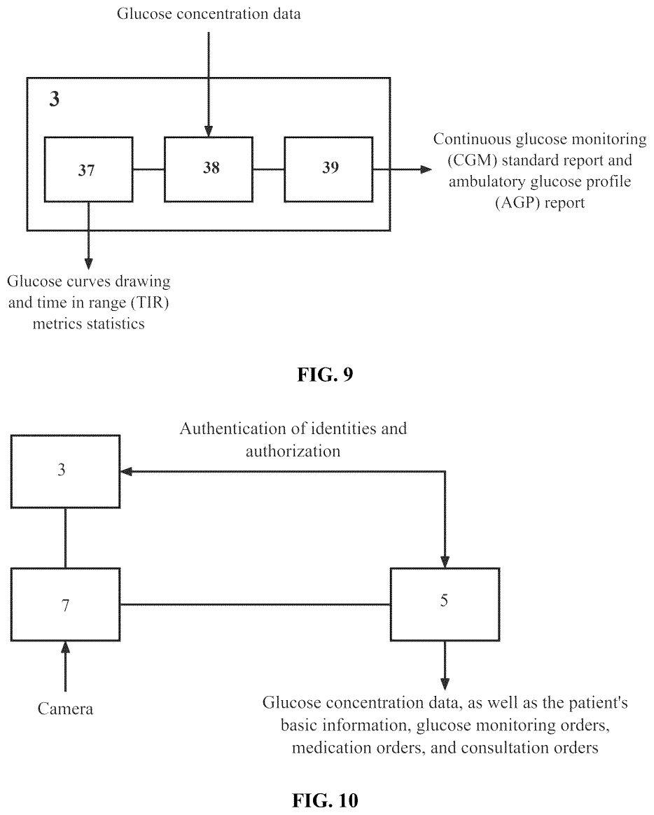

Further, as shown in FIG. 9, the patient user interface 3 of the present embodiment further comprises:
a drawing module 37, configured to perform glucose curves drawing and TIR metrics statistics according to the glucose concentration data of the patient;
an adjusting module 38, configured to correct the glucose curves and the TIR metrics based on the glucose concentration data; and
a generation module 39, configured to generate a CGM standard report and an ambulatory glucose profile report for the glucose concentration data.

Furthermore, the glucose curves include a single-day glucose curve graph, a multi-day continuous glucose monitoring thumbnail graph, and a multi-day quartile graph.

Furthermore, the glucose curves and the TIR metrics diagram show the glucose status of the patient through different colors.

In the present embodiment, the patient user interface 3 illustrated in FIG. 9 is configured to: analyze collected glucose data and user-specific information; generate glucose curves; and calculate time in range (TIR) metrics, thereby enabling users to comprehensively assess the monitoring status of glucose. In this process, the glucose curves and the TIR index may be corrected according to the glucose data, thereby obtaining more accurate glucose monitoring results, which facilitates subsequent diagnosis and treatment of the condition.

The time in range (TIR) in the target range is an important indicator for measuring glucose control, which may present a percentage of the glucose value within a normal numerical range (70-180 mg/dL). The higher the proportion within the range of normal values of TIR, the better the control of glucose. In general, it is recommended that TIR should be greater than 70 in medicine. Clinical empirical studies have also found that for every 10% increase in TIR, glycated hemoglobin (HbA1c) decreases by 0.5%, and the higher the proportion of TIR, it will help to delay diabetes-related complications, such as retinopathy and so forth. In addition, TIR may also provide more information than traditional point data, such as the duration of hyperglycemia and hypoglycemia. In general, to increase the proportion of time spent on TIR, it is medically recommended to consider reducing the duration of hypoglycemia as a safer practice. In addition, the TIR frame sets the home page of the patient user interface, and during the wearing period of the CGM device, the user can conveniently check the TIR to master the overall sugar control condition at any time, for example, the TIR box does not reach the standard and can be quickly strained. For example, discussing adjustment practices with the care team, if it has reached the standard, it may also allow users to relax moderately and avoid excessive tension.

Further, the patient user interface 3 further comprises an alarm module, configured to send alarm information to the patient user interface 3 when a glucose value of the patient deviates from a healthy glucose value range, and such as high blood sugar alarms, imminent low blood sugar alarms, low blood sugar alarms and even TIR abnormal alarms, etc., can timely attract the attention of the target user; and at the same time, the cloud server 4 includes an alarm module that can send an alarm message to the patient's authorized remote caregiver.

### Embodiment 2

An embodiment of the present disclosure discloses a transcutaneous glucose monitoring system 1, as shown in FIG. 10, comprising:
a partnership establishment module 7, configured to authorize a connection by scanning, via a camera, barcode information associated with the CGM device, or configured to transmit an invitation link to the patient user interface via a third-party platform (such as LINE, EMail or Messenger), thereby completing authentication of a patient identity and a partner identity as well as authorization; and
a partner user interface 5, configured to query glucose concentration data, as well as the patient's basic information, glucose monitoring orders, medication orders, and consultation orders, based on the patient's authorization

Wherein, steps for establishing a partner relationship via barcode scanning include:
a) on the partner user interface 5, the “Add Relationship” is clicked and the bar code scanning mode is entered;
b) on the patient user interface 3, a barcode containing a unique identifier is generated;
c) on the partner user interface 5, the barcode is scanned using a camera or barcode scanner, and the parsed identifier is sent to a server;
d) the server verifies the validity of the identifier and returns basic information of the partner user interface 5 to the patient user interface 3;
e) after confirming the partner information on the partner user interface 3, “Confirm Join” is clicked to request the server to establish a data sharing relationship;
f) the server formally binds the two accounts and allows the partner user interface 3 to access the continuous glucose monitoring data of the partner user interface 5; and
g) the server synchronizes the data according to the predetermined condition and triggers a notification mechanism to prompt abnormal glucose data or important changes.

Steps for establishing the partner relationship through the invitation via the third-party platform include:
a). an invitation request is sent by the patient user interface 3 to the server;
b).the server generates a dedicated invitation link according to the invitation request, the invitation link containing a unique identifier;
c). the barcode is scanned by a camera or barcode scanner on the patient user interface 3 and the parsed identifier is sent to the server;
d). the invitation link is transmitted on the patient user interface 3 via at least one third-party platform to the partner user interface 5;
e). the invitation link is clicked on the partner user interface 5 and the request is passed to the server;
f). the server verifies the validity of the unique identifier and prompts the partner user interface 5 to authenticate the identity with the account or establish a new account;
g). after obtaining consent on the partner user interface 5, the server establishes a data-sharing relationship between the account of the partner user interface 3 and the account of the partner user interface 5; and
h). the server synchronizes the glucose data from the partner user interface 3 to the partner user interface 5, and triggers a notification mechanism based on predetermined conditions.

In the present embodiment, the process of establishing a partner relationship is simplified through bar code scanning or the third-party platform invitation, thereby making account binding more efficient. Additionally, data security is enhanced by adopting a unique identifier and a server verification mechanism, which improves the security of data transmission. In addition, the system has a real-time notification mechanism, and when the patient's glucose data is abnormal, a warning may be automatically sent to the partner user interface 5 to ensure real-time response. In order to improve the flexibility of use, the system allows the partner user interface 5 to set the visual range and the access time length of the data, and support the multi-user mode, so that the patient user interface 3 of the plurality of patients may establish a data sharing relationship with the same partner user interface 5, thereby improving the convenience and efficiency of remote care.

Further, in the transdermal glucose monitoring system 1 of the present embodiment, after receiving the authorization application of the partner user interface 5 on the patient user interface 3, the patient selects the data that may be consulted and authorizes the data by using the digital signature for subsequent data query.

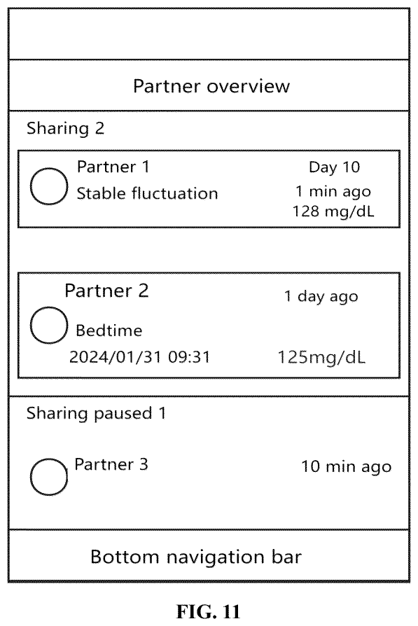

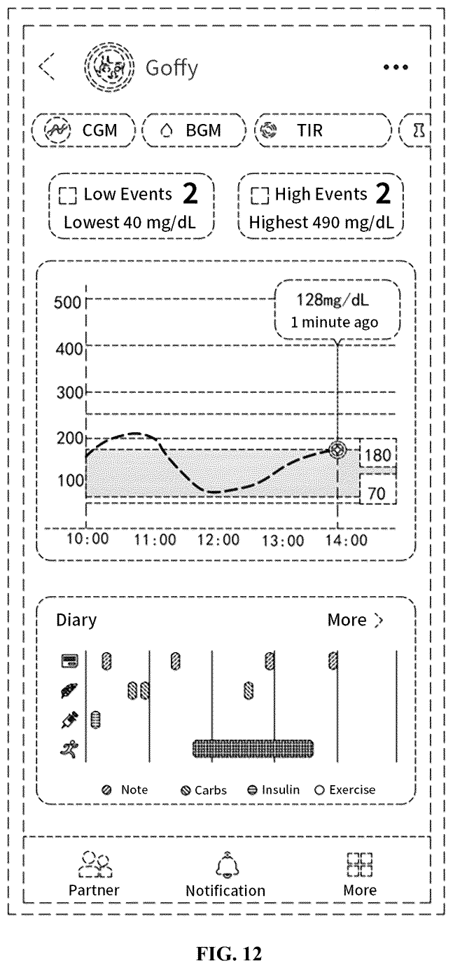

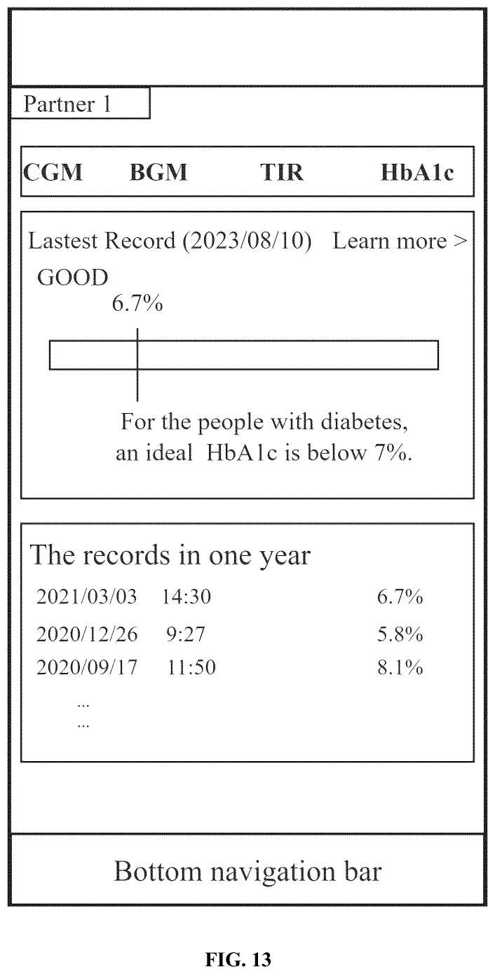

Further, as shown in FIG. 11 and FIG. 12, the partner user interface 5 in the present embodiment includes:
an alert module, configured to monitor glucose fluctuations of multiple partners to enable timely and effective interventions
an update module, configured to prioritize and observe real-time updates of a specific partner's glucose monitoring status;
a glucose display module, configured to acquire current glucose monitoring statuses of multiple partners and infer potential causes of fluctuations based on trends in glucose changes; and
a blood glucose display module, configured to acquire the most recent glucose measurement records.

In FIG. 11, the glucose situation of each partner may be sorted according to the data update time, and the user of the partner user interface 5 may preferentially see the glucose monitoring situation of a certain partner; wherein, the use of CGM data enables quick evaluation of the patient's current monitoring condition and allows for the deduction of fluctuation causes from historical glucose trends, whereas blood glucose data provides access to the most recent blood glucose measurements. In the present embodiment, the partner user interface 5 monitors the blood glucose status of the partner by integrating the blood glucose monitor (BGM) and the CGM device, thereby achieving a more diversified and accurate approach to information acquisition.

As shown in FIG. 12, further, the partner user interface 5 further comprises an annotation classification module, configured to record glucose anomaly events and their occurrence times, mark a range of glucose anomalies, and convert a marked content onto a timeline for comparing glucose fluctuations, thereby inferring potential causes of the glucose fluctuations. Wherein, the event categories may be distinguished by different colors. After the annotation classification module converts the marked content to the time axis, the patient and the partner client can intuitively compare the glucose change with a specific event (such as diet, exercise or pressure, etc.), thereby adjusting living habits or treatment plans, and improving personalized management. The event category is distinguished by different colors, so that a user can quickly identify and pay attention to a specific type of abnormality, such as low blood sugar, hyperglycemia, or fluctuation related to a specific activity, thereby further improving monitoring efficiency and identifying potential risks in real time. In another embodiment, the annotation classification module may also be disposed at the patient user interface 3.

Further, in the present embodiment, the partner user interface 5 further comprises a glycated hemoglobin display module, which is configured to map a patient's glycated hemoglobin value onto a color-coded chart, thereby enabling direct identification of the specific range within a commonly recognized standard system where the patient's glycated hemoglobin result falls. A visualization engine configured to render therapeutic outcome representations while reducing cognitive load, thereby enabling intuitive evaluation of glycemic control progression in patients.

The embodiments in this specification are described in a progressive manner, each embodiment focuses on differences from other embodiments, and the same or similar parts between the embodiments refer to each other.

The above description of the disclosed embodiments enables those skilled in the art to implement or use the present disclosure. Various modifications to these embodiments will be readily apparent to those skilled in the art, and the generic principles defined herein may be implemented in other embodiments without departing from the spirit or scope of the present disclosure. Thus, the present disclosure may not be limited to the embodiments shown herein but is to be accorded the widest scope consistent with the principles and novel features disclosed herein.

What is claimed is:

1. A transdermal glucose monitoring system, comprising:
a continuous glucose monitoring (CGM) device, configured to monitor glucose concentration in subcutaneous interstitial fluid of a patient;
a patient user interface, configured to acquire information of the CGM device, receive glucose concentration data, and display a warm-up progress indicator of the CGM device; and
a cloud server, configured to complete data sharing and authorize access to data;
wherein a user interface of the patient user interface comprises:
a top information section, configured to provide remaining usage days, a real-time glucose value and trend indicators of the CGM device, and visually indicate glycemic status through color variations or graphical symbols;
a glucose trend line section, configured to graphically display glucose trend data over a past predetermined number of hours;
a daily monitoring record overview chart section, configured to display daily glucose trend patterns in the patient over a multi-day period;
a time in range (TIR) distribution chart section, configured to provide statistical data for glucose management, display a percentage of time for patient's glucose values within a target glucose range, and visually represent a temporal distribution of glucose levels via color-coded bar charts; and
a bottom navigation bar, configured to provide functional buttons, to enable the patient to quickly access different functions of an application program.

2. The transdermal glucose monitoring system according to claim 1, wherein the CGM device comprises:
a glucose sensor, being implanted subcutaneously into the patient, configured to monitor the glucose concentration in subcutaneous interstitial fluid of the patient;
a temperature sensor, configured to monitor environmental temperature;
a glucose measurement unit, configured to apply a voltage to the glucose sensor to measure the glucose concentration;
a battery, configured to supply power; and
a wireless communication unit, configured to transfer data.

3. The transdermal glucose monitoring system according to claim 1, wherein the patient user interface comprises:
a user interface, configured to provide a warm-up status display, a glucose data display and a report analysis function;
a wireless communication module, configured to receive the glucose concentration data transmitted by the CGM device;
a memory, configured to store the glucose concentration data, trend analysis, health records of the patient, and calibration data;
at least one processor, configured to perform a plurality of functional instructions;
a warm-up control unit, configured to provide the warm-up progress indicator and completion time of the CGM device via the at least one processor; and
a glucose data processing unit, configured to, upon completion of warm-up, continuously receive and process the glucose concentration data via the at least one processor, generate real-time glucose reading values, dynamic glucose fluctuation curves and glucose management reports.

4. The transdermal glucose monitoring system according to claim 3, wherein the warm-up control unit comprises:
a time setting module, configured to enable the at least one processor to provide a scheduled warm-up completion time for the CGM device based on a warm-up preset time;
a response module, configured to enable the at least one processor to monitor a power-on action of the CGM device and to respond thereto; and
a time recording module, configured to cause the at least one processor to record a power-on time and an elapsed warm-up time of the CGM device.

5. The transdermal glucose monitoring system according to claim 3, wherein the warm-up control unit comprises:
a power check module, configured to monitor remaining battery levels of the CGM device and the patient user interface, compare the remaining battery levels with a preset activation threshold, to determine whether to initiate power-on and warm-up procedures; and
a time setting module, configured to cause the at least one processor to generate a scheduled completion time of warm-up for the CGM device based on a warm-up preset time.

6. The transdermal glucose monitoring system according to claim 1, wherein the patient user interface comprises:
an annotation module, configured to record medication status, diet status, and exercise status of the patient, and obtain user information of the patient according to a log record;
a warm-up progress display module, configured to display warm-up progress of the CGM device; and
a glucose display module, configured to display current glucose concentration data and glucose value fluctuations according to different types of trend indicators in a block design.

7. The transdermal glucose monitoring system according to claim 6, wherein a display mode of the warm-up progress display module comprises: elapsed warm-up time and/or scheduled completion time of warm-up.

8. The transdermal glucose monitoring system according to claim 1, wherein the patient user interface further comprises a calibration unit, the calibration unit comprises:
a blood glucose data acquisition module, configured to receive blood glucose measurement values obtained from a patient via a blood glucose monitor (BGM), and be autonomously input by the patient or automatically transmitted to the patient user interface;
a determining module, configured to analyze validity of blood glucose data and confirm whether the CGM device operates normally; and
a calibration unit, configured to write the blood glucose data into the patient user interface when the blood glucose data is valid and the CGM device operates normally.

9. The transdermal glucose monitoring system according to claim 1, wherein the patient user interface further comprises:
a drawing module, configured to perform glucose curves drawing and TIR metrics statistics according to the glucose concentration data of the patient;
an adjusting module, configured to correct glucose curves and TIR metrics based on the glucose concentration data; and
a generation module, configured to generate a CGM standard report and an ambulatory glucose profile report for the glucose concentration data.

10. The transdermal glucose monitoring system according to claim 9, wherein the glucose curves comprise a single-day glucose curve graph, a multi-day continuous glucose monitoring thumbnail graph, and a multi-day quartile graph.

11. The transdermal glucose monitoring system according to claim 9, wherein the glucose curves and a TIR metrics diagram show a glucose status of the patient through different colors.

12. The transdermal glucose monitoring system according to claim 1, wherein the patient user interface further comprises an alarm module, configured to send alarm information to the patient user interface when a glucose value of the patient deviates from a healthy glucose value range.

13. A transdermal glucose monitoring system, comprising:
a partnership establishment module, configured to authorize a connection by scanning, via a camera, barcode information associated with the CGM device, or configured to transmit an invitation link to a patient user interface via a third-party platform, wherein authentication of a patient identity and a partner identity as well as authorization are completed; and
a partner user interface, configured to query glucose concentration data, as well as patient's basic information, glucose monitoring orders, medication orders, and consultation orders, based on the authorization.

14. The transdermal glucose monitoring system according to claim 13, wherein the partner user interface comprises:
an alert module, configured to monitor glucose fluctuations of a plurality of partners to enable timely and effective interventions;
an update module, configured to prioritize and observe real-time updates of a specific partner's glucose monitoring status;
a glucose display module, configured to acquire current glucose monitoring statuses of the plurality of partners and infer potential causes of fluctuations based on trends in glucose changes; and
a blood glucose display module, configured to acquire most recent glucose measurement records.

15. The transdermal glucose monitoring system according to claim 13, wherein the partner user interface further comprises an annotation classification module, configured to record glucose anomaly events and occurrence times of the glucose anomaly events, mark a range of glucose anomalies, and convert a marked content onto a timeline for comparing glucose fluctuations, wherein potential causes of the glucose fluctuations are inferred.

16. The transdermal glucose monitoring system according to claim 13, wherein the partner user interface further comprises a glycated hemoglobin display module, wherein the glycated hemoglobin display module is configured to map a patient's glycated hemoglobin value onto a color-coded chart to enable direct identification of a range within a commonly recognized standard system where a patient's glycated hemoglobin result falls.
